(19) United States
(12) Patent Application Publication
(10) Pub. No.: US 2022/0098177 A1
(43) Pub. Date: Mar. 31, 2022

(54) POLYMER COMPOSITIONS COMPRISING COMPOUNDS DERIVED FROM BIOLOGY

(71) Applicant: Zymergen Inc., Emeryville, CA (US)

(72) Inventors: John J. McNamara, El Sobrante, CA (US); Carol A. Koch, San Gabriel, CA (US); Michael William Angus MacLean, Oakland, CA (US); Stephen Lo, San Francisco, CA (US); Ke Li, Wilmington, DE (US); Jason P. Safko, Redwood City, CA (US); Md Nazim Uddin, Berkeley, CA (US); Jenny Shao, South San Francisco, CA (US); Joachim C. Ritter, San Rafael, CA (US)

(73) Assignee: Zymergen Inc., Emeryville, CA (US)

(21) Appl. No.: 17/418,165

(22) PCT Filed: Dec. 30, 2019
(86) PCT No.: PCT/US2019/068979

§ 371 Date: Jun. 24, 2021

Related U.S. Application Data
(60) Provisional application No. 62/872,617, filed on Jul. 10, 2019.
(60) Provisional application No. 62/786,962, filed on Dec. 31, 2018.

(51) Int. Cl. C07D 405/14; C08G 59/50; C07D 241/08; C07D 487/14

(52) U.S. Cl. CPC C07D 405/14; C07D 487/14; C07D 241/08; C08G 59/5073

(57) ABSTRACT

A compound comprises a moiety selected from a cyclic dimer of a first and a second amino acid or a 2.5-diketopiperazine made from an amino acid. The moiety can be produced by fermentation. The compound further includes a polymerizable group. Additionally, the disclosure includes a method for preparing a resin comprises reacting the compound comprising the foregoing moiety and polymerizable group with a reagent.

### CROSS-REFERENCE TO RELATED APPLICATIONS

This application claims the benefit of U.S. provisional application No. 62/786,962, filed Dec. 31, 2018, and of U.S. provisional application No. 62/872,617, filed Jul. 10, 2019, both of which are hereby incorporated by reference in their entireties.

### FIELD OF THE DISCLOSURE

The present disclosure relates generally to the area of biologically derived compounds comprising polymerizable groups. More specifically, this disclosure relates to novel epoxy compositions that are comprised of amino acids, flavanones, flavones, benzophenones, amines, and heterocycles. Said epoxy compositions can undergo bio-triggered degradation for debonding of adhesives, coatings, and composites. Components of the epoxy compositions can be derived by biology through fermentation.

### BACKGROUND

Epoxy resins, because of their excellent mechanical properties, good adhesion properties to various substrates, the minimum cure shrinkage characteristics, are widely used in engineering coatings, bonding or the like. The most widely used are bisphenol A (BPA) type epoxy resins. Bisphenol A type epoxy resins having an epoxy group that, upon polymerization with amine hardeners yields amine bonds and a plurality of hydroxyl groups resulting in additional functionality and hydrogen bonding in the polymer compound, and, together with the aromatic structure of bisphenol A can significantly improve the mechanical properties of thermoset epoxy compounds.

However, in commonly used bisphenol A epoxy resins, the toxicity of BPA itself makes unsuitable as a starting material for applications in environmentally sensitive, biological, medical, or pharmaceutical fields that include polymer synthesis. Moreover, bisphenol A epoxy resin is generally considered non-degradable and therefore, this epoxy resin can cause environmental pollution.

### SUMMARY

In a first aspect, a compound comprises a moiety selected from (i) a cyclic dimer of a first and a second amino acid, (ii) a flavanone, (iii) a flavone, (iv) a benzophenone, or (v) a combination of the foregoing. The compound further includes a polymerizable group.

In a second aspect, a method for preparing a resin comprises reacting the compound comprising the foregoing moiety and polymerizable group with a reagent.

In a third aspect, a polymer curative comprises a moiety selected from (i) a cyclic dimer of a first and a second amino acid, (ii) a heterocycle, (iii) an amine, or (v) a combination of the foregoing. The polymer curative can further include at least two polymerizable groups.

In a fourth aspect. The foregoing compound according or the foregoing polymer curative are used in an adhesive, a composite, a coating, an electronic device, an energy storage device, or an energy generation device.

In a fifth aspect, a method for manufacturing an adhesive, a composite, a coating, an electronic device, an energy storage device, or an energy generation device comprises applying the foregoing compound or the foregoing polymer curative in the assembly of the adhesive, the composite, the coating, the electronic device, the energy storage device, or the energy generation device.

### DETAILED DESCRIPTION

This written description uses examples to disclose the embodiments, including the best mode, and also to enable those of ordinary skill in the art to make and use the invention. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

Note that not all of the activities described above in the general description or the examples are required, that a portion of a specific activity may not be required, and that one or more further activities may be performed in addition to those described. The order in which activities are listed is not necessarily the order in which they are performed.

In this specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Benefits, other advantages, and solutions to problems have been described herein with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range.

As a stated in the Summary, a compound comprises a moiety selected from (i) a cyclic dimer of a first and a second amino acid, (ii) a flavanone, (iii) a flavone, (iv) a benzophenone, or (v) a combination of the foregoing. In addition, the compound can include a polymerizable group.

A polymerizable group includes groups that form homopolymers or copolymers. In a first embodiment, the polymerizable group can form predominately homopolymers, meaning that the compound A forms polymers symbolized as -(A-A-A)ₓ- wherein x is an integer. These groups are defined as homopolymerizable. Examples of such groups are unsaturated groups, such as vinyl and allyl groups, oxiranes (ethylene oxides or epoxides), aziridines (ethylene imines), oxetanes. In another embodiment, the polymerizable group is copolymerizable, i.e., a second compound B is required to form polymers -(A-B-A-B)ₓ- wherein x is an integer. Examples of such groups are carboxylic acids, hydroxyl groups, amino groups, thiol groups; and examples for the respective copolymer monomer would be diols or diamines, diacids, diacid anhydrides, isocyanates, di-isocyanates.

In one embodiment, the polymerizable group can be selected from a vinyl group, an allyl group, an epoxy group, or a combination thereof.

In another embodiment, at least 35 wt %, such as at least 40 wt %, at least 45 wt %, at least 50 wt %, at least 55 wt %, at least 60 wt %, at least 65 wt %, at least 70 wt %, at least 75 wt %, at least 80 wt %, or at least 85 wt % of the compound is comprised by the moiety. In another embodiment, not more than 98 wt %, such as not more than 96 wt %, not more than 95 wt %, not more than 94 wt %, not more than 92 wt %, or not more than 90 wt % of the compound are comprised by the moiety. In yet one further embodiment, the moiety of the compound has weight percentage in the range between 30 wt % to 99.5 wt %, such as 40 wt % to 98 wt %, or even 50.5 wt % to 96 wt %.

In yet one further embodiment, at least 60 wt %, at least 65 wt %, at least 70 wt %, at least 75 wt %, at least 80 wt %, at least 85 wt %, or at least 88 wt % of the compound are comprised by the sum of weight percentages of the moiety and the polymerizable group. In another embodiment, not more than 99.9 wt %, such as not more than 99 wt %, not more than 98 wt %, not more than 96 wt %, not more than 94 wt %, not more than 92 wt %, not more than 90 wt %, not more than 85 wt %, or not more than 80 wt % of the compound are comprised by the sum of weight percentages of the moiety and the polymerizable group. In yet one further embodiment, the sum of weight percentages of the moiety and the polymerizable group can range between 55 wt % to 99.99 wt %, such as 65 wt % to 99 wt %, or 75 wt % to 98 wt %.

In another embodiment, the compound can be selected from:

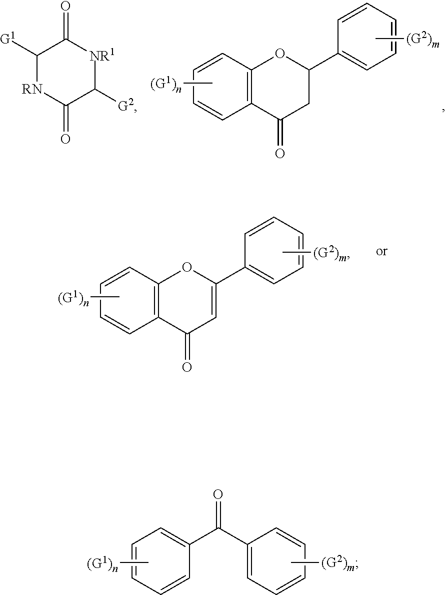

wherein R and R¹ independently for each occurrence are selected from hydrogen, alkyl, halogenated alkyl, alkoxy-alkyl, or any combination thereof;

wherein G¹ and G² independently for each occurrence can be selected from

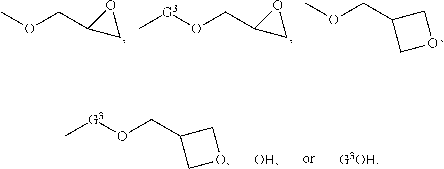

In another embodiment, G¹ and G² independently for each occurrence can be selected from

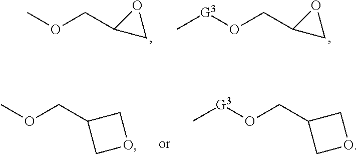

The group G³ can be an alkylene, an arylene, or an alkylarylene. In one embodiment, G³ can be methylene, ethylene, n-propylene, isopropylene, n-butylene, 2-methylpropylene, n-pentylene, 2-methylbutylene, 2,3-dimethylpropylene, 1,4-phenylene, methylene-phenylene, para-methylene-phenylene, ethylene-phenylene, or para-ethylene-phenylene. In the foregoing structures, wherein n and m independently for each occurrence are integers selected from 1 through 5.

In one further embodiment, the compound can be selected from the following list of compounds or an subset thereof:

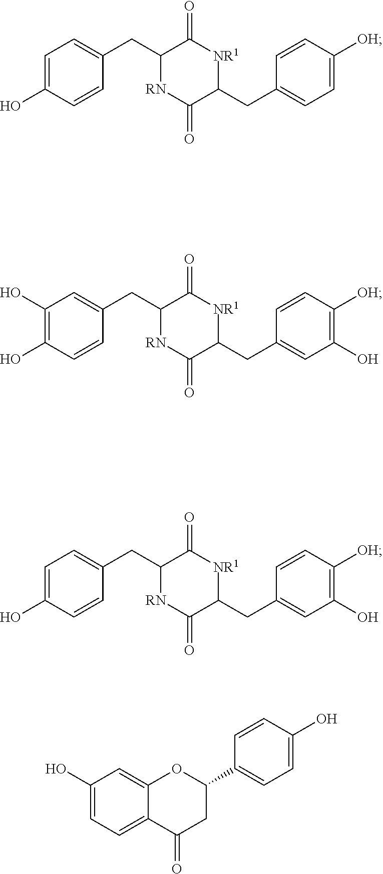

or its enantiomer;

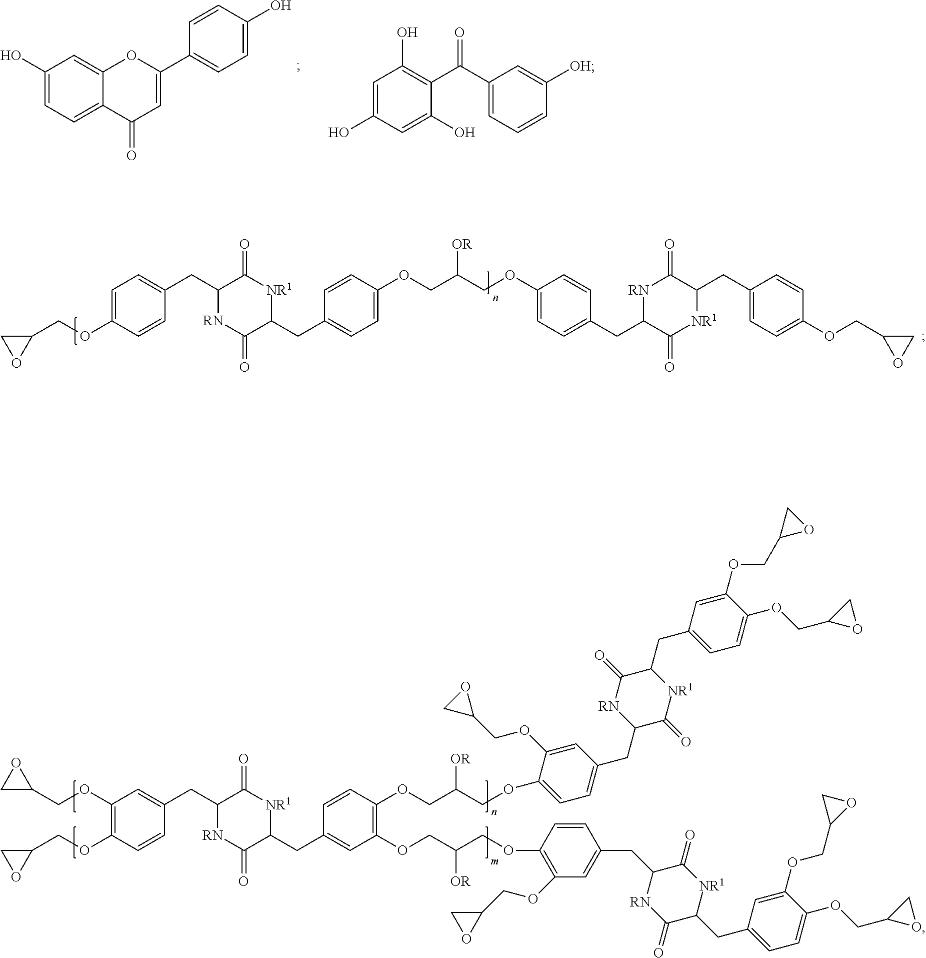

wherein R and R¹ independently for each occurrence are selected from hydrogen, alkyl, halogenated alkyl, alkoxy-alkyl, or any combination thereof, n and m are integers including zero and n+m>0; such as

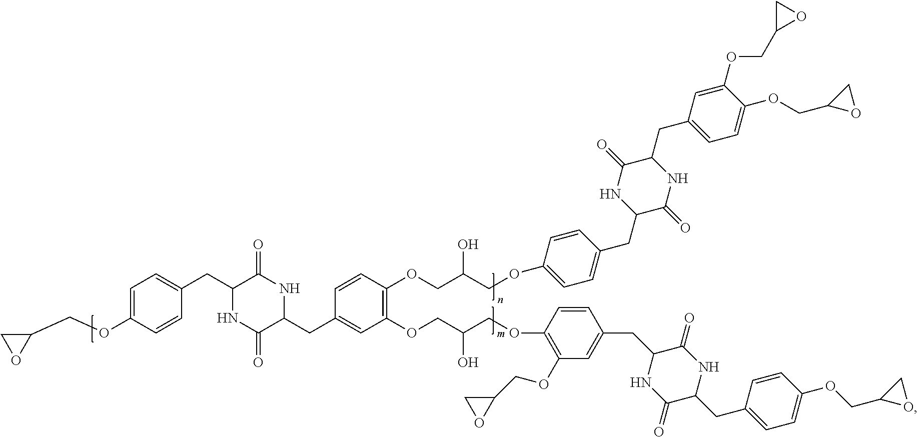

wherein n and m are integers including zero and n+m>0;

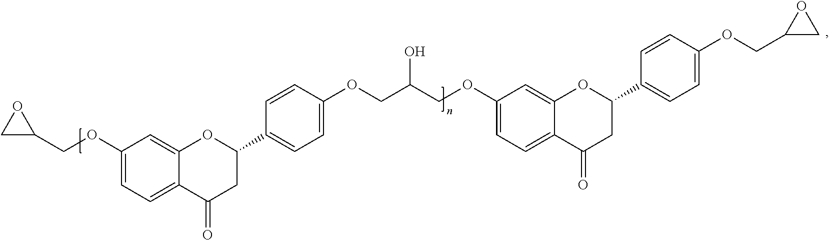

wherein n is an integer including zero;

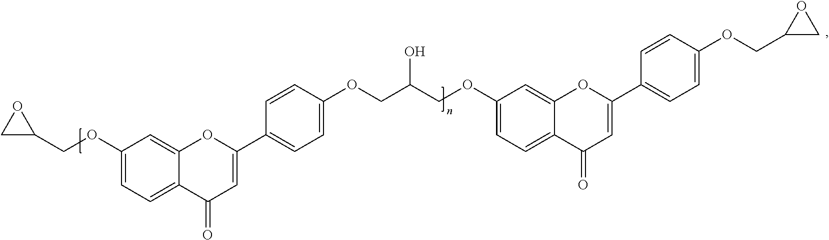

wherein n is an integer including zero; or

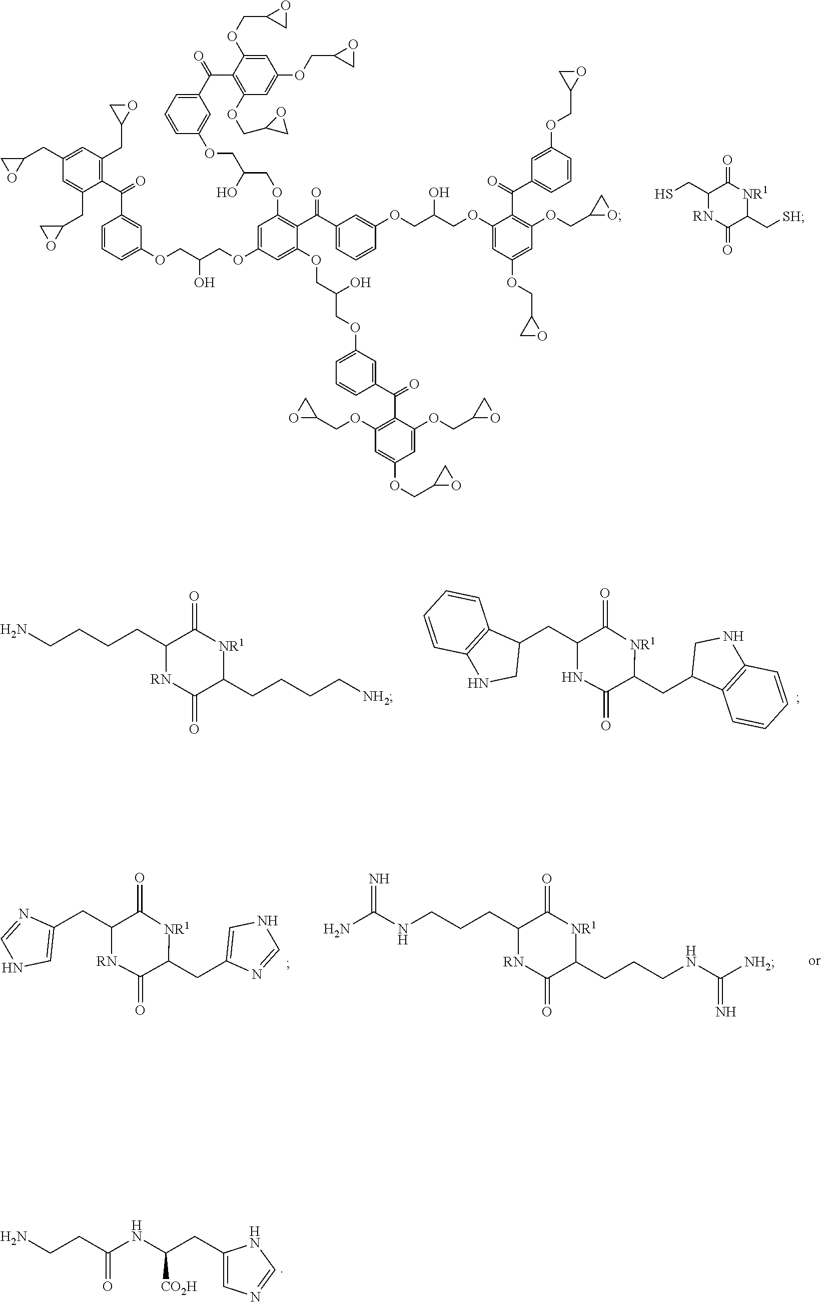

In addressing compounds comprising groups R and R¹, these groups can include hydrogen, alkyl, alkoxyalkyl. For example R and R¹ can be, independently for each occasion, hydrogen or C₁ to C₂₀ straight or branched alkyl chains, such as methyl, ethyl, n-propyl, 2-propyl, 1-butyl, 2-butyl, 2-methylpropyl, pentyl, 2-methylbutyl, 2,2-dimethylpropyl, hexyl, 2-methylpentyl, 3-methylpentyl, 2,2-dimethylbutyl, 2,3-dimethylbutyl, heptyl, 2-methylhexyl, 3-methylhexyl, 2,2-dimethylpentyl, 2,3-dimethylpentyl, 2,4-dimethylpentyl, 3,3-dimethylpentyl, 3-ethylpentyl, 2,2,3-trimethylbutyl, octyl, 2-methylheptyl, 3-methylheptyl, 4-methylheptyl, 5-methylheptyl, 6-methylheptyl, 2-ethylhexyl, 3-ethylhexyl, 4-ethylhexyl, 2,3-dimethylhexyl, 2,4-dimethylhexyl, 2,5-dimethylhexyl, 3,4-dimethylhexyl, 3,5-dimethylhexyl, 4,5-dimethylhexyl, 2-propylpentyl, nonyl, decyl; undecyl, dodecyl, tridecyl, tetradecyl, pentadecyl, hexadecyl, heptadecyl, octadecyl, nonadecyl, and icosyl.

In one embodiment, the compound based on amino acid dimers can be selected from:

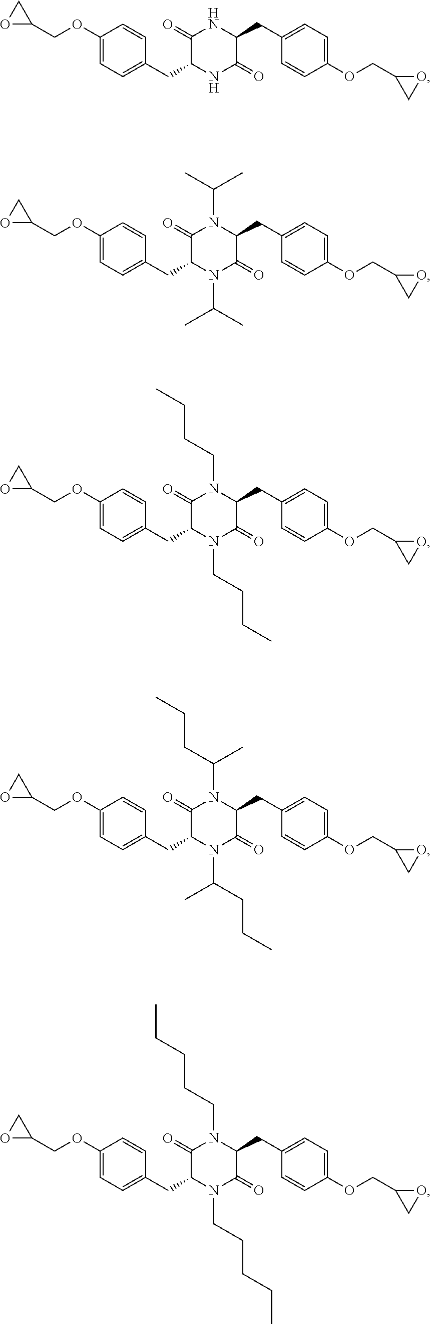 [SMILES: O=C1N[C@H](Cc2ccc(OCC3CO3)cc2)C(=O)N[C@H]1Cc1ccc(OCC2CO2)cc1 ; CC(C)N1C(=O)[C@H](Cc2ccc(OCC3CO3)cc2)N(C(C)C)C(=O)[C@H]1Cc1ccc(OCC2CO2)cc1 ; CCCCN1C(=O)[C@H](Cc2ccc(OCC3CO3)cc2)N(CCCC)C(=O)[C@H]1Cc1ccc(OCC2CO2)cc1 ; CCCC(C)N1C(=O)[C@H](Cc2ccc(OCC3CO3)cc2)N(C(C)CCC)C(=O)[C@H]1Cc1ccc(OCC2CO2)cc1 ; CCCCCN1C(=O)[C@H](Cc2ccc(OCC3CO3)cc2)N(CCCCC)C(=O)[C@H]1Cc1ccc(OCC2CO2)cc1]

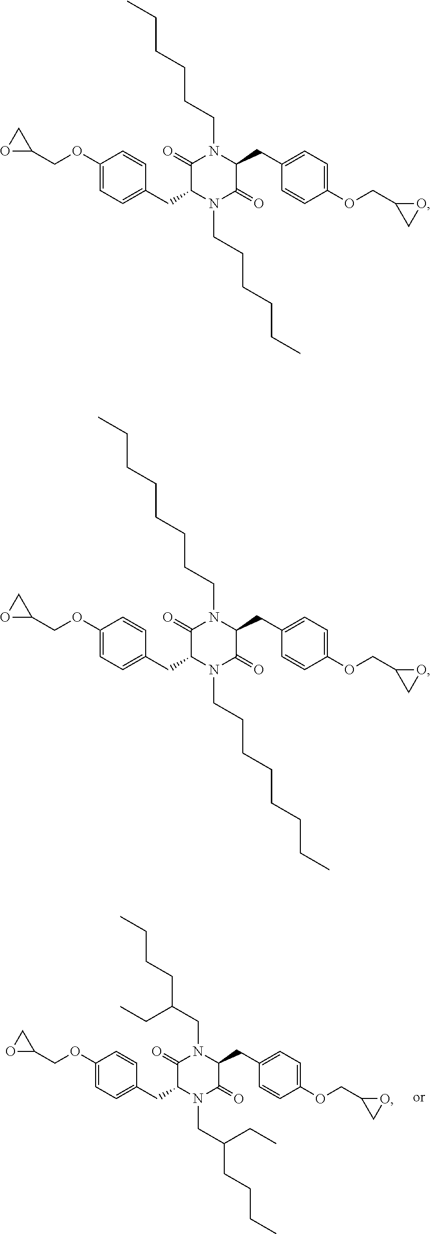 [SMILES: CCCCCCN1C(=O)[C@H](Cc2ccc(OCC3CO3)cc2)N(CCCCCC)C(=O)[C@H]1Cc1ccc(OCC2CO2)cc1 ; CCCCCCCCN1C(=O)[C@H](Cc2ccc(OCC3CO3)cc2)N(CCCCCCCC)C(=O)[C@H]1Cc1ccc(OCC2CO2)cc1 ; CCCCC(CC)CN1C(=O)[C@H](Cc2ccc(OCC3CO3)cc2)N(CC(CC)CCCC)C(=O)[C@H]1Cc1ccc(OCC2CO2)cc1]

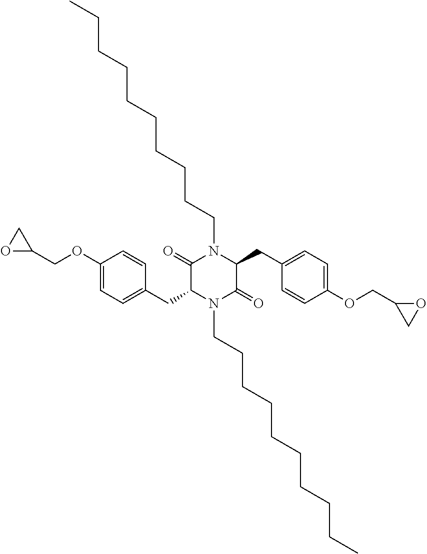 [SMILES: CCCCCCCCCCN1C(=O)[C@H](Cc2ccc(OCC3CO3)cc2)N(CCCCCCCCCC)C(=O)[C@H]1Cc1ccc(OCC2CO2)cc1]

Addressing synthesis of some of the compounds, those can be achieved for example by:

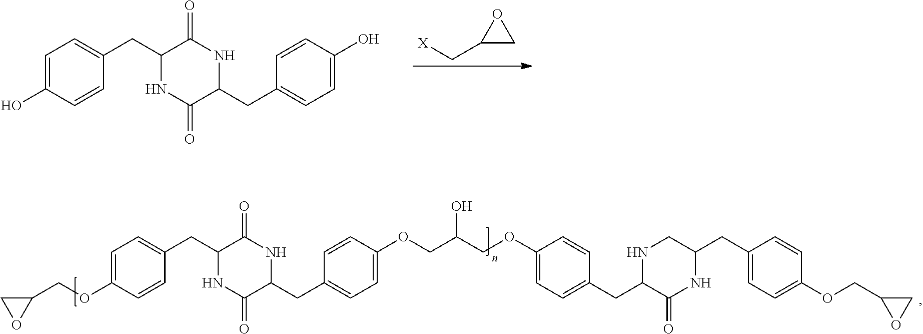

wherein X can be Cl or Br.

Another example for modifying an amino acid dimer with a polymerizable group is as follows:

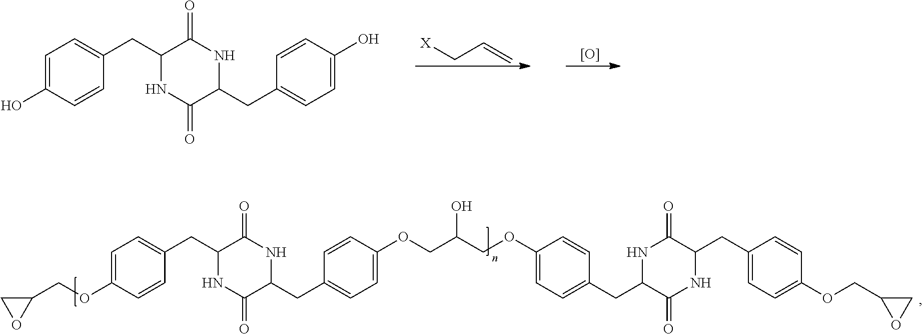

wherein X is Cl or Br and [O] is an oxidizing agent, n is an integer including zero.

The epoxidation reaction can be stoichiometric, i.e., one mole of epichlorohydrin or epibromohydrin per mole of hydroxy groups in the moiety. Alternatively, epoxidation can be conducted to a lesser degree, wherein the ratio of moles of hydroxy group over moles of reagent can range from 20:1 to 0.9:1, such as from 15:1 to 1:1, 10:1 to 1:1, or 5:1 to 1:1. This is true for any other reagent that renders the moiety polymerizable, such as allyl halides or vinyl halides.

The oxidation reaction in the above scheme serves to render epoxides from unsaturated organic groups. In one embodiment, an oxidation reaction is omitted to allow the unsaturated group to be the polymerizable group. Here too, all hydroxyl groups or a fraction thereof can react to give a polymerizable group. Oxidation reagents can be peroxides, percarboxylic acids, percarboxylic esters, percarboxylic salts, chlorine, hypochlorous acid, or hypochlorites.

In one embodiment, the amino acid dimers can be selected from

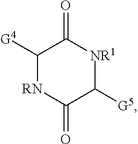

wherein R and R¹ are selected independently for each occasion from hydrogen, alkyl, halogenated alkyl, or alkoxylalkyl; G⁴ and G⁵ are selected independently from the group of —NH₂, —SH, —NHC(NH)NH₂, -G⁶NH₂, -G⁶NHG⁷, -G⁶NG⁷G⁸, -G⁶SH, -G⁶NHC(NH)NH₂ wherein G⁶ is selected from an alkylene, an arylene, an alkylarylene; G⁷, G⁸ are selected from alkyl or aryl. It follows that dimers of different amino acids are contemplated within the scope of the disclosure. For example, a dimer of cysteine, homocysteine, or one of each are contemplated herewith.

For example, structure contemplates within the scope of the present disclosure are epoxidized dimers of hydroxylated phenylalanines. As can be seen in the structures below, the epoxy groups can be symmetrically located in ortho, meta, or para positions, but also asymmetrical locations, i.e., ortho-meta, ortho-para, or meta-para are contemplated within this disclosure.

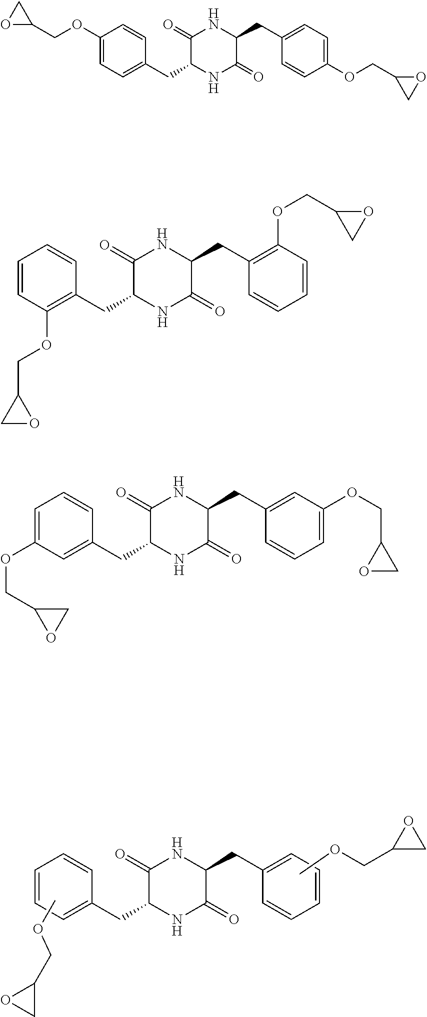

In one further aspect, a polymer curative comprises a moiety selected from (i) a cyclic dimer of a first and a second amino acid, (ii) a heterocycle, (iii) an amine, or (v) a combination of the foregoing. The polymer curative further includes at least two polymerizable groups.

In one embodiment, the at least two polymerizable groups can, independent for each occurrence, be selected from a vinyl group, an allyl group, an epoxy group, or a combination thereof. In yet another embodiment, the moiety comprises at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, or at least 85 wt % with respect to the weight of the polymer curative. In one embodiment, not more than 92 wt %, such as not more than 90 wt %, not more than 88 wt %, not more than 86 wt %, not more than 85 wt %, not more than 80 wt %, or not more than 75 wt % of the polymer curative is comprised of the moiety.

In another embodiment, the sum of moiety and the at least two polymerizable groups comprises at least 60 wt %, at least 65 wt %, at least 70 wt %, at least 75 wt %, at least 80 wt %, at least 85 wt %, or at least 88 wt % of the polymer curative. In yet another embodiment, the sum of moiety and the at least two polymerizable groups comprises not more than 99 wt %, such as not more than 98 wt %, not more than 96 wt %, not more than 95 wt %, not more than 90 wt %, or not more than 85 wt % of the polymer curative.

In one further embodiment, for moieties comprising amino acid dimers, the first and the second amino acids can be selected independently from cysteine, lysine, ornithine, histidine, arginine, or tryptophan.

In yet one further embodiment, the polymer curative includes:

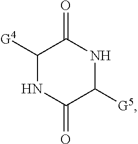

wherein
G⁴ and G⁵ can be selected independently for each occurrence from the group of —NH₂, —SH, —NHC(N)NH₂, -G⁶NH₂, -G⁶NHG⁷, -G⁶NG⁷G⁸, -G⁶SH, -G⁶NHC(NH)NH₂. In the foregoing structure, G⁶ can be selected from an alkylene, an arylene, an alkylarylene; G⁷, G⁸ are selected from alkyl or aryl.

In one embodiment, for heterocycles as the moiety, the heterocycle can be selected from histamine, tryptamine, or carnosine. In case of the moiety being an amine, the amine can be selected from putrescine, cadaverine, spermine, spermidine, norspermine, norspermidine, or agmatine.

When it comes to applications of the foregoing compound or the foregoing polymer curative, they can be used in an adhesive, a composite, a coating, an electronic device, an energy storage device, or an energy generation device. Accordingly, the present disclosure includes a method for manufacturing an adhesive, a composite, a coating, an electronic device, an energy storage device, or an energy generation device.

In one further embodiment, the foregoing compound has a bio-based carbon content of at least 10%, such as at least 15%, at least 20%, at least 25%, at least 30%, or at least 35% as determined by ASTM D6866. Bio-based carbon content as defined herein is the percentage of carbons from renewable or biogenic sources, such as plants or animals over the total number of carbons in the compound.

For example, the following compound is prepared from bio-sourced tyrosine and petrochemically epichlorohydrin:

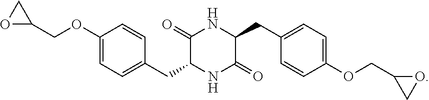 [SMILES: O=C1N[C@H](Cc2ccc(OCC3CO3)cc2)C(=O)N[C@H]1Cc1ccc(OCC2CO2)cc1]

Then, 16 carbon atoms are bio-based and 6 carbon atoms are petrochemically sourced. Upon analysis according to ASTM D6866, this compound has a bio-based carbon content of 16/(16+6)=72.7%.

The method includes applying one of the foregoing compounds or a foregoing polymer curative in the assembly of the adhesive, the composite, the coating, the electronic device, the energy storage device, or the energy generation device.

In summary, this disclosure relates polymer precursors such as epoxy compositions comprised epoxy resins and curatives as exemplified above. One concept of this disclosure relates to bio-based epoxy resins comprised of cyclic dipeptides, flavanones, flavones, and benzophenones. Another aspect of this invention relates to epoxy resins that are made by epoxidizing a bio-based compound comprising hydroxyl or amine groups. Epoxidation can be complete or partial, such as in the range of 1:0.1 to 1:1 (eq/eq) OH/epoxidation reagent. An aspect of this disclosure relates to cyclic dipeptides for epoxy resins. Such dimers can be made of tyrosine, dopamine, or tyrosine and dopamine. Epoxidation reagents can be epichlorohydrin and epibromohydrin. Another option relates to epoxidation reagents that are allyl halides and oxidants. Another option relates to epoxidation reagents that are unsaturated acids and oxidants. Polymer curatives include epoxy curatives comprised of cyclic amino acid dimers, heterocycles, and amines. Another aspect of this disclosure relates to cyclic dipeptides for epoxy curatives that are comprised of cysteine, lysine, histidine, arginine, tryptophan. Another aspect of this disclosure relates to heterocycle epoxy curatives comprised of histamine, tryptamine, and carnosine putrescine. Another aspect of this disclosure relates to amine epoxy curatives comprised of agmatine, cadaverine, spermine, spermidine, norspermine, or norspermidine. Another aspect of this disclosure relates to epoxy compositions used in adhesives. Another aspect of this disclosure relates to epoxy compositions used in composites. Another aspect of this disclosure relates to epoxy compositions used in coatings. Another aspect of this disclosure relates to epoxy compositions used applications involving electronics, energy storage, energy generation, civil engineering, architectural, industrial, and transportation. Another aspect of this disclosure relates to epoxy compositions comprising methanone having anti-oxidation properties. Another aspect of this disclosure relates to epoxy compositions comprising carnosine having oxygen scavenging properties for species like peroxides, superoxides, and singlet oxygen. Another aspect of this disclosure relates to epoxy compositions of that undergo bio-degradation. Another aspect of this disclosure relates to epoxy compositions that are produced by way of bio-engineered yeast, bacteria, and fungi using fermentation.

Without limiting the scope of the present disclosure, the following list represents exemplary embodiments:
Item 1. A compound comprising a moiety selected from:
   (i) a cyclic dimer of a first and a second amino acid,
   (ii) a flavanone,
   (iii) a flavone,
   (iv) a benzophenone, or
   (v) a combination of the foregoing;
and a polymerizable group.
Item 1(a) The compound according to item 1, wherein the moiety comprises a cyclic dimer of a first and a second amino acid.
Item 2. The compound according to item 1, wherein the polymerizable group is selected from a vinyl group, an allyl group, an epoxy group, or a combination thereof.
Item 3. The compound according to item 1, wherein at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, or at least 85 wt % comprises the moiety.
Item 4. The compound according to item 1 or 1(a), wherein at least 60 wt %, at least 65 wt %, at least 70 wt %, at least 75 wt %, at least 80 wt %, at least 85 wt %, or at least 88 wt % comprises a sum of the moiety and the polymerizable group.
Item 5. The compound according to item 1 selected from:

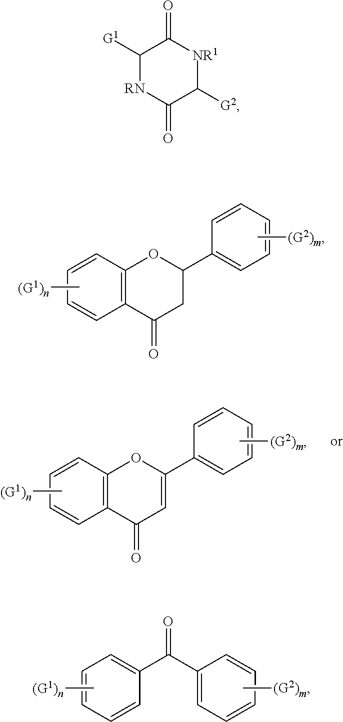

wherein R and R¹ independently for each occurrence are selected from hydrogen, alkyl, halogenated alkyl, alkoxy-alkyl, or any combination thereof; wherein G¹ and G² independently for each occurrence are selected from

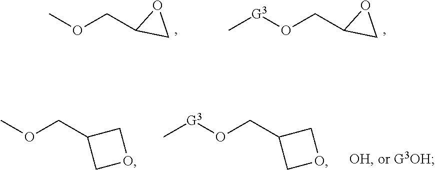

wherein G³ is an alkylene, an arylene, or an alkylarylene;
wherein n and m independently for each occurrence are integers selected from 1 through 5.
Item 6. A method for preparing a resin comprising:
reacting a compound according to item 1 with a reagent.
Item 7. The method according to item 6, wherein the compound comprises OH groups and the reacting is initiated at a ratio of moles of OH groups per moles of reagent ranging from 10:1 to 1:1.
Item 8. The method according to item 6, wherein the reagent is selected from epichlorohydrin, epibromohydrin, allyl halides, vinyl halides, unsaturated acids, or any combination thereof.
Item 9. The method according to item 8, wherein the reagent is selected from allyl halides, vinyl halides, unsaturated acids, or any combination thereof; and the method further includes adding an oxidant.
Item 10. The method according to item 9, wherein the oxidant is selected from chlorine, hypochlorous acid, a peroxycarboxylic acid, a peroxycarboxylate, a peroxyphthalate, or a combination thereof.
Item 11. A polymer curative comprising a moiety selected from:
   (i) a cyclic dimer of a first and a second amino acid,
   (ii) a heterocycle,
   (iii) an amine, or
   (v) a combination of the foregoing; and
at least two polymerizable groups.
Item 12. The polymer curative according to item 11, wherein the at least two polymerizable groups independent for each occurrence are selected from a vinyl group, an allyl group, an epoxy group, or a combination thereof.
Item 13. The polymer curative according to item 11, wherein at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, or at least 85 wt % with respect to the weight of the polymer curative comprises the moiety.
Item 14. The polymer curative according to item 11, wherein at least 60 wt %, at least 65 wt %, at least 70 wt %, at least 75 wt %, at least 80 wt %, at least 85 wt %, or at least 88 wt % comprises a sum of the moiety and the at least two polymerizable groups.
Item 15. The polymer curative according to item 11, wherein the first and the second amino acids are selected independently from cysteine, lysine, omithine, histidine, arginine, tryptophan, tyrosine, or dopamine.
Item 16. The polymer curative of item 11 comprising:

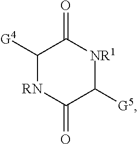

wherein
R and R¹ are selected independently for each occasion from hydrogen, alkyl, halogenated alkyl, or alkoxyalkyl;
G⁴ and G⁵ are selected independently from the group of —NH₂, —SH, —NHC(NH)NH₂, -G⁶NH₂, -G⁶NHG⁷, -G⁶NG⁷G⁸, -G⁶SH, -G⁶NHC(NH)NH₂; wherein G⁶ is selected from an alkylene, an arylene, an alkylarylene; G⁷, G⁸ are selected from alkyl or aryl.
Item 17. The polymer curative according to item 11, wherein the heterocycle is selected from histamine, tryptamine, or carnosine.
Item 18. The polymer curative according to item 11, wherein the amine is selected from putrescine, cadaverine, spermine, spermidine, norspermine, norspermidine, or agmatine.
Item 19. The compound according to item 1 or the polymer curative according to item 11 used in an adhesive, a composite, a coating, an electronic device, an energy storage device, or an energy generation device.
Item 20. A method for manufacturing an adhesive, a composite, a coating, an electronic device, an energy storage device, or an energy generation device comprising
applying a compound according to item 1 or a polymer curative according to item 11 in the assembly of the adhesive, the composite, the coating, the electronic device, the energy storage device, or the energy generation device.

### EXPERIMENTALS

### Synthesis of Tyrosine Dimer

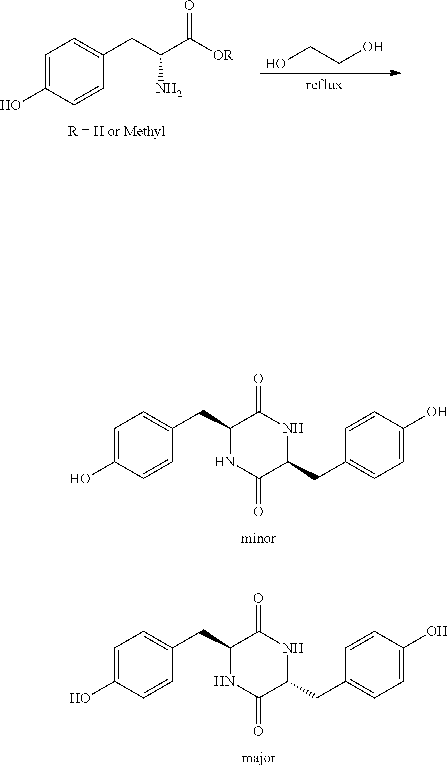

In a 3 L two-neck round bottom flask equipped with magnetic stirrer and overhead condenser, 200 g of Tyr-OH and 800 ml of ethylene glycol were mixed and the flask was placed in silicon oil bath. The oil bath was heated to 190° C. and the reaction mixture was stirred for 7 h. The conversion of starting material was followed up by HPLC. After 7 h the reaction mixture was cooled down to room temperature and the precipitated solid was filtered and washed with ethanol (2×200 ml). The solid was then dried in vacuum oven and used as is for the next step. (Yield: 64%)

### Synthesis of 4-hydroxy-proline dimer

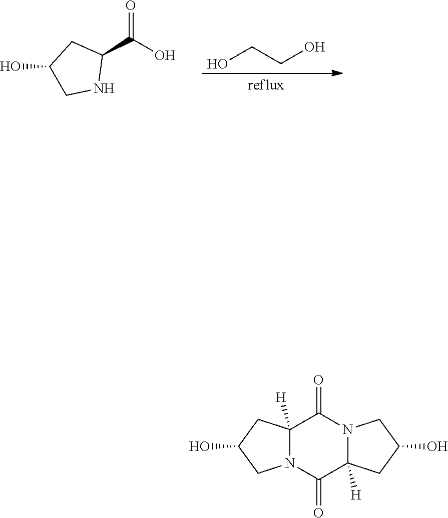

In a two-neck 1 L round bottom flask equipped with magnetic stirrer and overhead condenser, 100 g of trans-4-hydroxy-L-proline and 200 ml of ethylene glycol were mixed and the flask was placed in silicon oil bath. The oil bath was heated to 190° C. and the reaction mixture was stirred for 7 h. After 7 h the reaction mixture was cooled down to room temperature and the precipitated solid was filtered and washed with acetone (2×100 ml). The solid was then dried in vacuum oven. Isolated 37.95 grams of product, 44% yield. NMR ¹H NMR (D₂O): 4.75 (d, 1H), 4.63 (d, 1H), 3.69 (d, 1H), 3.537 (d, 1H), 2.33 (d, 1H), 2.20 (d, 1H)

Stepwise synthesis of Tyrosine dimer. (This route is applicable for dimers from different amino acids.)

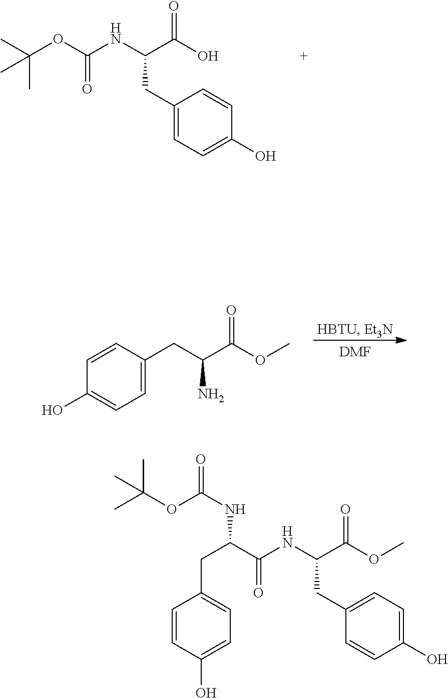

### Step 1: Preparation of (S)-methyl 2-((R)-2-((tert-butoxycarbonyl)amino)-3-(4-hydroxyphenyl) propanamido)-3-(4-hydroxyphenyl)propanoate

A 1 L reactor equipped with a magnetic stirrer, temperature probe and nitrogen inlet was charged with ((S)-2-((tert-butoxycarbonyl)amino)-3-(4-hydroxyphenyl)propanoic acid (33.2 g, 118 mmol), (S)-methyl 2-amino-3-(4-hydroxyphenyl)propanoate (20 g, 102 mmol), Hexafluorophosphate Benzotriazole Tetramethyl Uronium (“HBTU,” v48.3 g, 127 mmol) and DMF (120 mL). The solution was stirred for 15 minutes and then cooled to 0° C. Triethylamine (42.6 mL, 306 mmol) was added to the mixture over 15 minutes. After the addition was completed, the cooling bath was removed and the reaction was stirred overnight. After 18 h, the HPLC of the aliquot showed complete conversion of the starting materials. 100 mL of water was slowly added to the reaction at 0° C. After stirring for 30 min, the mixture was diluted with EtOAc (150 mL) and the layers were separated. The organic layer was washed with aqueous sodium carbonate (10%, 3×50 mL), and finally with brine (50 mL). The organic layer was then dried over anhydrous sodium sulfate, filtered, and concentrated to dryness to afford the desired product as a thick oil. The product was used in the next step without further purification.

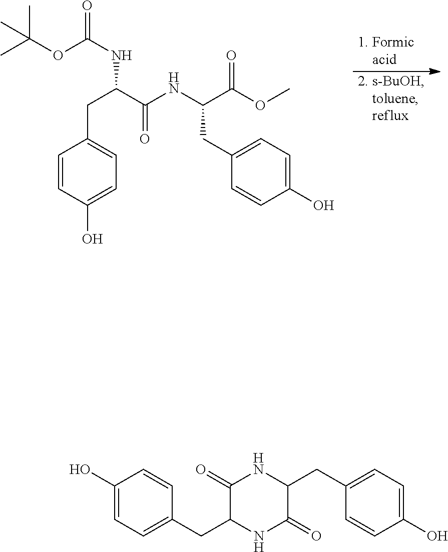 [SMILES: COC(=O)[C@H](Cc1ccc(O)cc1)NC(=O)[C@H](Cc1ccc(O)cc1)NC(=O)OC(C)(C)C ; O=C1NC(Cc2ccc(O)cc2)C(=O)NC1Cc1ccc(O)cc1]

### Step 2: Preparation of 3,6-bis(4-hydroxybenzyl)piperazine-2,5-dione

A 3 L single-neck reactor was charged with (S)-methyl 2-((R)-2-((tertbutoxycarbonyl)amino)-3-(4-hydroxyphenyl)propanamido)-3-(4-hydroxyphenyl)propanoate (42 g, 91.6 mmol) and formic acid (420 mL) and the mixture was stirred at ambient temperature for 5 h and the formic acid and s-butanol were removed under reduced pressure. The residue was dissolved in sec-butanol (1600 mL) and toluene (400 mL) and the solution was refluxed for 3 hours. The reaction was monitored by HPLC and, after the reaction was completed, the reaction mixture was concentrated to yield the crude material as an off-white solid. The crude material was dissolved in 5% NaOH in water at 5° C., extracted with 250 ml Ethyl Acetate, and then the aqueous layer was acidified to pH 3 by the slow addition of 10% HCl (aq). The solid material was separated by filtration, washed with water, and dried under vacuum. The solid was suspended in 200 ml of acetonitrile and filtered again and dried to get a white solid as a pure product. (Yield—22 g, 73%). NMR ¹H NMR (DMSO): 9.20 (s, 1H), 7.76 (s, 1H), 6.84 (d, J=8.4 Hz, 2H), 6.67 (d, J=8.5 Hz, 2H), 3.85 (s, 1H), 2.55-2.51 (m, 1H), 2.12 (d, J=6.6 Hz, 1H)

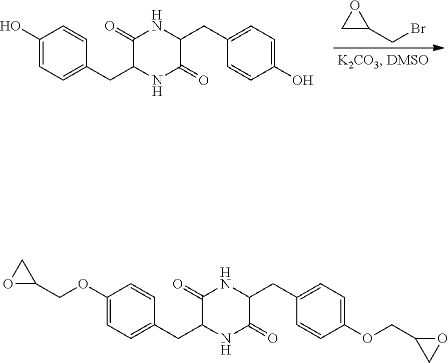

### Step 3: Preparation of 3,6-bis(4-(oxiran-2-ylmethoxy)benzyl)piperazine-2,5-dione

A 1 L single-neck reactor was charged with 3,6-bis(4-hydroxybenzyl)piperazine2,5-dione (2 g, 6.13 mmol) and DMSO (30 mL) and the mixture was stirred at ambient temperature for 30 minutes in order to allow the starting materials to dissolve. Potassium carbonate (3.4 g, 24.52 mmol) was added and the stirring was continued for 30 minutes. Epibromohydrin (1.6 mL, 18.40 mmol) was then added and the reaction mixture was stirred for 2 days at room temperature. The reaction mixture was filtered to remove the solids and the solid was rinsed with DMSO (20 mL). The filtrate solution obtained was slowly poured into ice cold water (100 ml). The solid was filtered, washed with water (100 ml), and dried under vacuum. The solid was suspended in 120 ml of acetonitrile, filtered, and the solid was dried under vacuum to yield an off-white solid. (Yield—1.9 g, 71%). NMR -GLC19575 ¹H NMR (DMSO): 7.86 (s, 1H), 6.95 (d, J=8.5 Hz, 2H), 6.87 (d, J=8.5 Hz, 2H), 4.31, 4.21 (m, 1H), 3.93 (s, 1H), 3.77 (dt, J=11.1, 6.2 Hz, 1H), 3.28 (d, J=2.6 Hz, 1H), 2.80 (t, J=4.6 Hz, 1H), 2.67 (s, 1H), 2.56 (dd, J=13.7, 4.4 Hz, 1H), 2.23 (dd, J=13.6, 6.0 Hz, 1H)

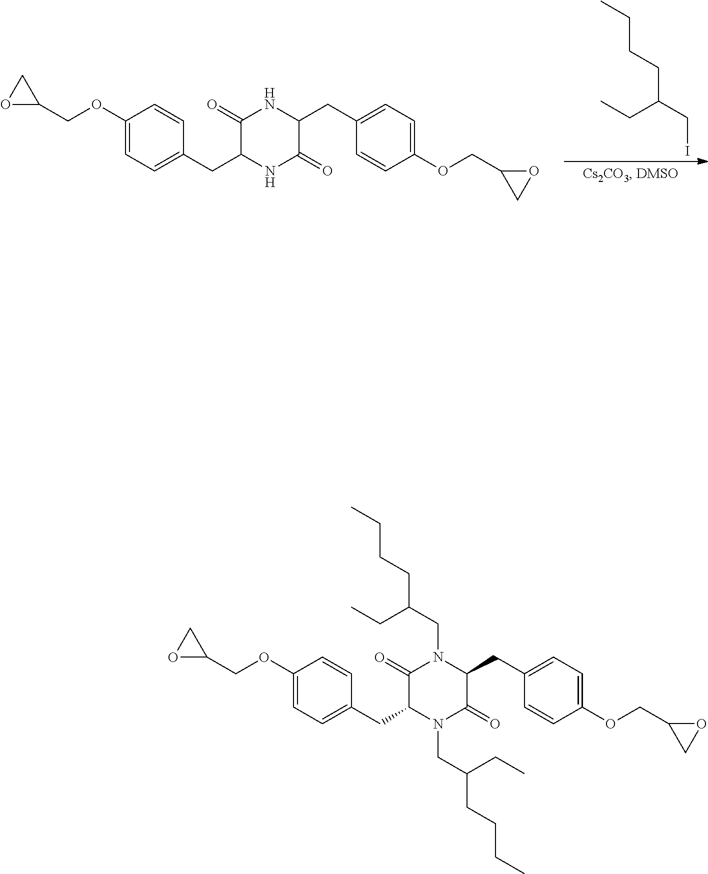

### Step 4-Preparation of 1,4-bis(2-ethylhexyl)-3,6-bis(4-(oxiran-2-ylmethoxy)benzyl)piperazine-2,5-dione (Compound 2B)

A 1 L single-neck reactor was charged with 3,6-bis(4-(oxiran-2-ylmethoxy)benzyl)piperazine-2,5-dione (10 g, 22.83 mmol) and dry DMSO (100 mL). The solution was stirred at ambient temperature for 30 minutes until a clear solution was obtained. Cesium carbonate (33.5 g, 102.7 mmol) was added and the stirring was continued for 30 minutes. 3-ethyl-1-iodohexane (14.4 mL, 79.9 mmol) was added to the mixture and the reaction mixture was stirred for 2 days. After 2 days, the HPLC of the aliquot showed more than 90% of the starting material was converted. The reaction mixture was filtered to remove the solids, the solids were rinsed with MTBE (100 mL), and the filtrate was slowly poured into 120 ml of ice cold water. The organic layer was separated and washed with 80 ml of water and 80 ml of brine. The solution was dried over sodium sulfate and concentrated under vacuum to yield the crude product as a yellow oil which was purified by column chromatography using EtOAc/hexane/Et₃N mixture. A yellow, clear oil was obtained. (Yield—2.6 g, 17%) NMR-GLC 20547 ¹H NMR (CDCl₃): 7.03 (d, J=8.4 Hz, 2H), 6.87 (d, J=8.3 Hz, 2H), 4.13 (t, J=9.2 Hz, 3H), 3.90 (dd, J=11.0, 5.5 Hz, 2H), 3.26-3.31 (m, 1H), 2.85 (t, J=4.5 Hz, 2H), 2.68-2.71 (m, 1H), 2.26-2.40 (m, 2H), 1.26, 0.98 (m, 9H), 0.84 (t, J=7.2 Hz, 3H), 0.78 (t, J=7.4 Hz, 2H), 0.71 (t, J=7.1 Hz, 2H).

### General Reaction for N-alkylation

The foregoing method was repeated with 2-ethylhexyliodide replaced for iodohexane, iodooctane, iododecane, iodododecane and the corresponding N-alkyl derivatives were obtained in yields between 17 and 53%.

For alkylation yielding the N-oleyl derivative, an Appel reaction procedure was implemented to prepare oleyl iodide. A round-bottom flask with stir bar was rendered dry by heating to 140° C. Based on a 10 gram scale of oleyl alcohol, 1.1 equivalent (“eq”) of PPh₃, 1.2 eq of iodine, and 1.1 eq of imidazole were weighted out and added to the round bottom flask which was then closed with a septa. 70 mL of DCM was added and the mixture was stirred vigorously. 10 grams of oleyl alcohol were added dropwise to the mixture. The mixture took on a yellow-orange color. The reaction was stirred for 2 days. After the reaction was confirmed to have reached completion by TLC, 20 mL of solid thiosulfate (10% w/v) was added. The organic layer was collected and washed twice with 20 ml of sodium thiosulfate, followed by washings with 30 ml of water and 30 ml of brine, dried over magnesium sulfate, then filtered over paper. The filtrate was concentrated in vacuo to form a white solid. The white solid was triturated with pentane, filtered over glass wool and concentrated in vacuo to form a yellow oil.

### Stepwise Synthesis of p-hydroxyphenyl-glycine dimer

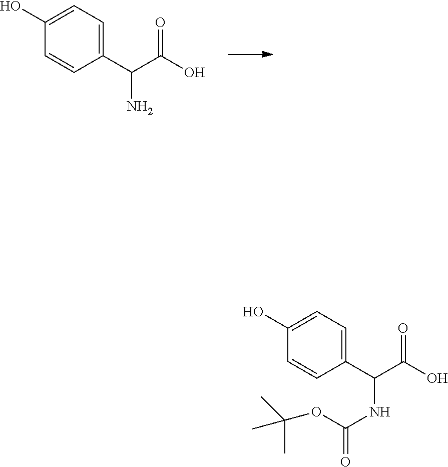 [SMILES: NC(C(=O)O)c1ccc(O)cc1 ; CC(C)(C)OC(=O)NC(C(=O)O)c1ccc(O)cc1]

(2R)-2-Amino-2-(4-hydroxyphenyl)acetic acid (1.00 eq, 1.00 g, 5.98 mmol) was dissolved in 1,4-Dioxane (24 mL), water (24 ml), and 12.5 ml of an aqueous 2M NaOH solution in a 100 ml 2 neck flask under nitrogen. Di-tert-butyl dicarbonate (1.00 eq, 1.31 g, 5.98 mmol) was added to the solution dropwise and the reaction was allowed to stir for 16 hours at room temperature. The reaction mixture was concentrated then acidified to pH 2 with 5M HCl, then extracted with ethyl acetate, washed with a 5% sodium carbonate solution, and brine. The organic layers were dried over magnesium sulfate, filtered, then concentrated in vacuo. 2-(tert-butoxycarbonylamino)-2-(4-hydroxyphenyl)acetic acid (1.08 g, 4.03 mmol, 67.36%% yield) was isolated as a pink tacky solid and used in the next step without further purification.

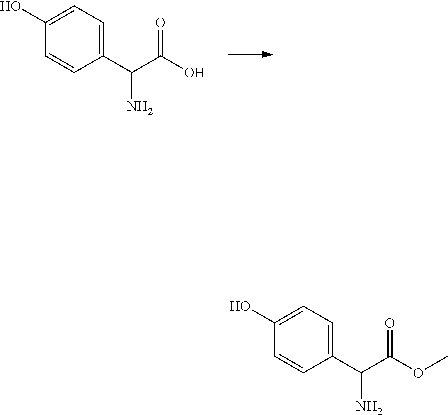

rac-(2R)-2-amino-2-(4-hydroxyphenyl)acetic acid (1.00 eq, 1.00 g, 5.98 mmol) was dissolved in 20 ml of 1.25M HCl in methanol and stirred at 70° C. for 3 hours and then the solvent was evaporated on a rotovap to yield 1.064 g of crude pink-white solid. This solid was washed with 250 ml of saturated sodium carbonate and extracted with ethyl acetate (4×100 ml). Isolated 0.366 g of product (33.766% yield).

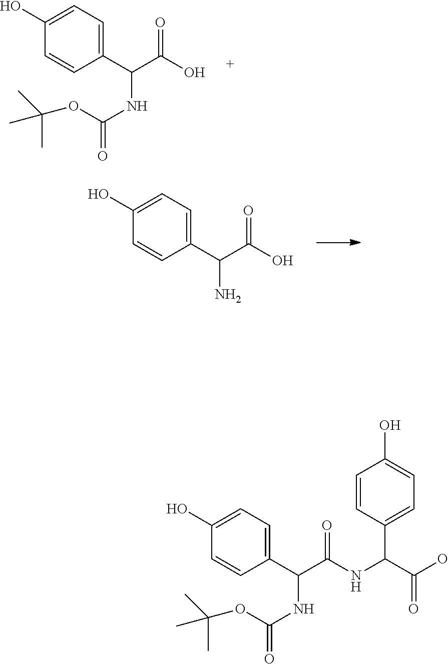

4-hydroxyphenyl-glycine methyl ester (4-HPG OMe), 4-HPG N-Boc, HBTU, and DMAc were added to a 25 ml 2 neck round bottom flask and stirred for 15 mins at room temperature under nitrogen. The reaction was cooled to 0° C. and trimethylamine (0.70 ml) was added dropwise over 15 mins and then allowed to stir overnight. The reaction was then quenched with 2 ml of ice cold water, stirred for 10 mins and extracted 3× with EtoAc (2 ml). The organic layers were washed with 5% sodium carbonate then brine, dried and concentrated.

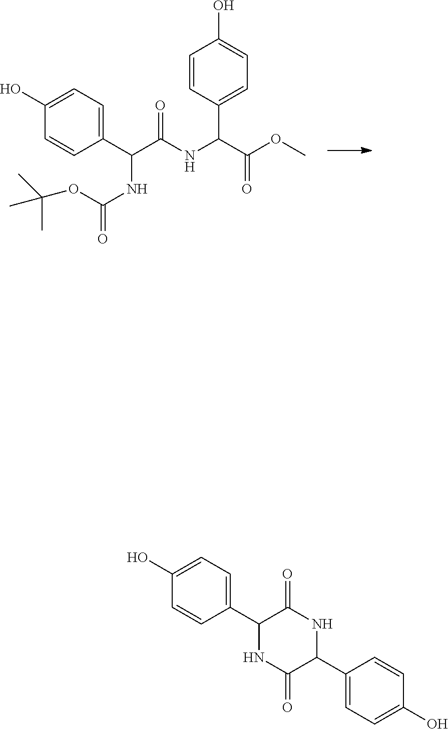

A 3 L single-neck reactor was charged with the foregoing dipeptide peptide (0.31 g) and formic acid (2.1 mL) and the mixture was stirred at ambient temperature for 5 hours and the formic acid was removed under reduced pressure by azeotropic distillation with toluene. The residue was dissolved in sec-butanol (7.5 mL) and toluene (2.5 mL) and the solution was refluxed for 3 hours. The reaction mixture was concentrated to yield the crude material as a yellow-white solid.

Performance Testing

Compound 2B was compared to Epon828 by formulating with several amine hardeners. Amine hardeners utilized were 1,4-diamino butane (DAB) or 1,13-diamino-4,7,10-trioxatridecane (TDD). When indicated, 2,4,6-Tris(dimethylaminomethyl)phenol was used as accelerator.

Formulations were approximately 1:1 equivalents of epoxy to amine. Gel Time was determined with a Rheometer, glass transition temperature T_{g} by Differential Scanning Calorimetry (DSC), decomposition temperature T_{d} by thermogravimetric analysis (TGA), coefficient of thermal expansion (CTE) by thermomechanical analysis (TMA), dynamic mechanical analysis (DMA) temperature sweep was used to determine temperature of maximal tan δ.

TABLE 1

| | | | Max. tanδ/ | | CTE (<T_{g})/ | CTE (>T_{g})/ |
|---|---|---|---|---|---|---|
| Epoxy resin | Hardener | T_{g}/° C. | ° C. | T_{d}/° C. | μm/min ° C. | μm/min ° C. |
| Epon828 | DAB + acc | 64 | 94.4 | 344 | 90 | 179 |
| 2B | DAB + acc | 51 | 76.3 | 362 | 102 | 198 |
| EPON828 | TDD | 73 | 64.4 | 354 | 64 | 190 |
| 2B | TDD | — | 64.4 | 360 | 55 | 196 |

As shown in Table 1, the performance of a bio-based compound 2B is comparable to the conventional epoxies.

Adhesive Properties

Lapshear testing was conducted according to ASTM 1004 and compared to Epon834 on cold rolled steel. Both epoxy resins were mixed with resorcinol diglycidyl ether (RDGE) in equal amounts as an additive.

TABLE 2

| Epoxy | | | | Lapshear/ |
|---|---|---|---|---|
| Resin | Hardener | Accelerator | additives | psi |
| 2B | TDD | DMP30 | RDGE | 2865 |
| Epon834 | TDD | DMP30 | RDGE | 2590 |

As shown in Table 2, the adhesive properties of the bio-based compound 2B is comparable to the conventional epoxy.

Water Absorption

Formulations of Epoxy resin, TDD amine, RDGE, and DMP-30 were prepared cured in a mold to give samples in form of strips (1×5×20 mm) after curing at 70° C. for 2 hours. Samples were weighed in vial, 10 g of distilled water was added. Samples were left at ambient conditions at the specified time of 1 to 29 days. Excess water was decanted and samples were blotted with tissue to remove excess water, then weighted again and weight gain calculated as shown in Table 3.

TABLE 3

| Epoxy | 1 d | 2 d | 8 d | 15 d | 21 d | 29 d |
|---|---|---|---|---|---|---|
| Epon828 Formulation | 2.30 | — | 4.30 | 5.02 | — | 6.24 |
| 2B Formulation | 2.07 | — | 5.40 | 6.35 | — | 6.87 |

As can be seen in Table 3, the water absorption of the bio-based epoxies is comparable to the conventional epoxy.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range.

What is claimed is:

1. A compound comprising a moiety, the moiety including
a cyclic dimer of a first and a second amino acid, and
a polymerizable group.

2. The compound according to claim 1, wherein the polymerizable group is selected from a vinyl group, an allyl group, an epoxy group, or a combination thereof.

3. The compound according to claim 1, wherein at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, or at least 85 wt % comprises the moiety.

4. The compound according to claim 1, wherein at least 60 wt %, at least 65 wt %, at least 70 wt %, at least 75 wt %, at least 80 wt %, at least 85 wt %, or at least 88 wt % comprises a sum of the moiety and the polymerizable group.

5. The compound according to claim 1 selected from: 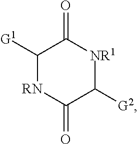
wherein R and R¹ independently for each occurrence are selected from hydrogen, alkyl, halogenated alkyl, alkoxyalkyl, or any combination thereof;
wherein G¹ and G² independently for each occurrence are selected from 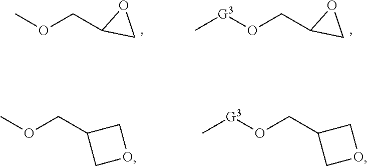
wherein G³ is an alkylene, an arylene, or an alkylarylene;
wherein n and m independently for each occurrence are integers selected from 1 through 5.

6. A method for preparing a resin comprising:
reacting a compound according to claim 1 with a reagent.

7. The method according to claim 6, wherein the compound comprises OH groups and the reacting is initiated at a ratio of moles of OH groups per moles of reagent ranging from 10:1 to 1:1.

8. The method according to claim 6, wherein the reagent is selected from epichlorohydrin, epibromohydrin, allyl halides, vinyl halides, unsaturated acids, or any combination thereof.

9. The method according to claim 8, wherein the reagent is selected from allyl halides, vinyl halides, unsaturated acids, or any combination thereof; and the method further includes adding an oxidant.

10. The method according to claim 9, wherein the oxidant is selected from chlorine, hypochlorous acid, a peroxycarboxylic acid, a peroxycarboxylate, a peroxyphthalate, or a combination thereof.

11. A polymer curative comprising a moiety the moiety including
a cyclic dimer of a first and a second amino acid, and
at least two polymerizable groups.

12. The polymer curative according to claim 11, wherein the at least two polymerizable groups independently for each occurrence are selected from a vinyl group, an allyl group, an epoxy group, or a combination thereof.

13. The polymer curative according to claim 11, wherein at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, or at least 85 wt % with respect to the weight of the polymer curative comprises the moiety.

14. The polymer curative according to claim 11, wherein at least 60 wt %, at least 65 wt %, at least 70 wt %, at least 75 wt %, at least 80 wt %, at least 85 wt %, or at least 88 wt % comprises a sum of the moiety and the at least two polymerizable groups.

15. The polymer curative according to claim 11, wherein the first and the second amino acids are selected independently from cysteine, lysine, omithine, histidine, arginine, tryptophan, tyrosine, or dopamine.

16. The polymer curative of claim 11 comprising: 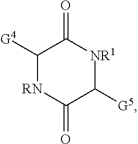
wherein
R and R¹ are selected independently for each occurrence from hydrogen, alkyl, or alkoxyalkyl;
G and G⁵ are selected independently from the group of —NH₂, —SH, —NHC(NH)NH₂, -G⁶NH₂, -G⁶NHG⁷, -G⁶NG⁷G⁸, -G⁶OH, -G⁶SH, -G⁶NHC(NH)NH₂;
wherein G⁶ is selected from an alkylene, an arylene, an alkylarylene; G⁷, G⁸ are selected from alkyl or aryl.

17. The polymer curative according to claim 11, wherein the first and the second amino acids are selected independently from cysteine, lysine, ornithine, histidine, arginine, tryptophan, tyrosine, or dopamine.

18. The polymer curative according to claim 11, selected from

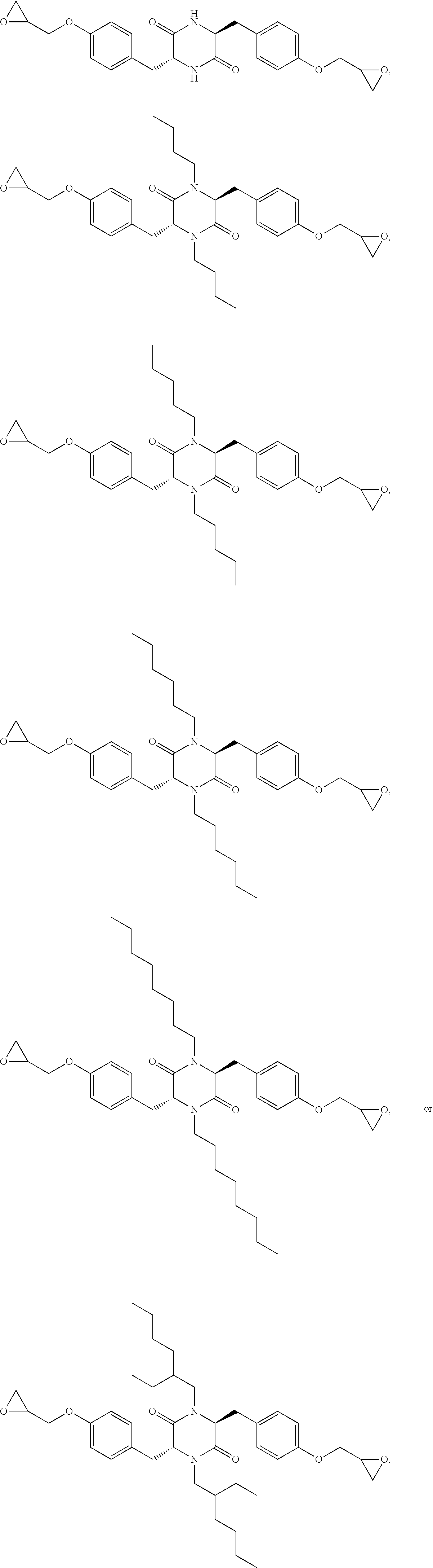

19. The polymer curative according to item 11 used in an adhesive, a composite, a coating, an electronic device, an energy storage device, or an energy generation device.

20. A method for manufacturing an adhesive, a composite, a coating, an electronic device, an energy storage device, or an energy generation device comprising
applying a compound according to claim 1.
